(19) United States
(12) Patent Application Publication
(10) Pub. No.: US 2021/0346819 A1
(43) Pub. Date: Nov. 11, 2021

(54) SYSTEM AND METHOD FOR FRACTIONAL DISTILLATION

(71) Applicant: ENTOURAGE BIOSCIENCES INC., Vancouver (CA)

(72) Inventors: Mike McCune, Vancouver (CA); Robert August, Vancouver (CA); Sazzad Hossain, Richmond (CA)

(21) Appl. No.: 17/277,786

(22) PCT Filed: Sep. 27, 2019
(86) PCT No.: PCT/CA2019/051391

§ 371 Date: Mar. 19, 2021

Related U.S. Application Data
(60) Provisional application No. 62/738,770, filed on Sep. 28, 2018.

(51) Int. Cl. B01D 3/32; B01D 3/42; B01D 5/00

(52) U.S. Cl. CPC B01D 3/32; B01D 5/006; B01D 3/42

(57) ABSTRACT

Cannabinoids can be extracted from plant material in the form of a crude oil. Such crude oils can be fractionally distilled to remove solvents and other volatiles used in the extraction process, terpenes, and isolate cannabinoids in the form of a cannabinoid concentrate. A system for fractional distilling to isolate cannabinoids can comprise a fractionating column, a condensing head and a thermal jacket for controlling the fluidity of the liquid distillate being isolated and collected.

### FIELD

Embodiments of this invention relate to glassware for fractional distillation and more specifically related to glassware for fractional distillation of extracts from cannabis plant material for producing a concentrate containing cannabinoids.

### BACKGROUND

The Cannabis sativa plant is plant species comprising Cannabis sativa, Cannabis indica, and Cannabis ruderalis, and is commonly used to produce hemp fibre, hemp oils, and also commonly used to produce tetrahydrocannabinol (THC) for recreational drug use, and cannabidiol (“CBD”) for medicinal purposes. Both THC and CBD are part of a group of chemicals known as cannabinoids. While not completed supported by clinical trials, pharmaceutical compositions containing cannabinoids have been developed to treat a variety of ailments, including treatments for nausea, vomiting, increasing appetite and treatment of chronic pain and muscle spasms.

With the recent trend towards the legalization of cannabis for medicinal and, in certain cases, recreational use, a complete industry surrounding the cultivation of cannabis, the sale of cannabis, and more specifically, pharmaceutical compositions specifically containing cannabinoids have been developed.

To manufacture pharmaceutical compositions containing cannabinoids, concentrates containing cannabinoids are isolated from cannabis plant materials or extracts using various isolation techniques, including factional distillation. The concentrates, once purified, can be made into a pharmaceutical composition suitable for consumption by patients by the addition of various chemical constituents.

Fractional distillation is a technique that is commonly used in chemistry and even taught in high school chemistry courses. Fractional distillation is a technique for separating individual components or constituents that make up a mixture or composition. Fractional distillation relies on the concept that each constituent making up the composition or mixture (made up of several different constituents) has a unique boiling or vaporization point that is different than the boiling point of the other constituents making up the composition. The composition is heated to a threshold temperature, causing each constituent to boil off or vaporize. Thus, as heat is applied to the composition, the constituent having the lowest boiling point would be the first constituent that would evaporate into vapors, and be separated from the remaining constituents still remaining in the composition. The first constituent or first fraction, is then condensed back into a liquid form and collected in a receiving flask. Most commonly, the vapors are condensed using a condenser unit that has a cold fluid, or a fluid having a temperature below that of the boiling point of the first constituent, running therethrough.

In typical and most simple fractional distillation systems, a vertical fractionating column interconnects the boiling flask or vessel and a condensing unit. The condensing can be fluidly connected directly to the vertical fractionating column, or can be interconnected to the vertical fractionating column with a head. Often, the condensing unit is positioned at an angle relative the vertical fractionating column, such that when the vapors of each constituent are condensed therein, the liquid constituents flow away from the vertical fractionating column and into a receiving flask or container.

As already known, the composition is continuously heated, to evaporate the first fraction, then to evaporate a subsequent second fraction, and so forth, until all of the composition is evaporated into its many individual constituents, which are collected in separate receiving flasks.

Typically, a factional distillation system for isolating cannabinoids from a cannabinoid containing concentrate comprises the following components: 1) a reaction vessel for containing the plant material extract; 2) fractionating columns for assisting with the separation of constituents having boiling points that are similar to one another; 3) a distillation head for functionally and fluidly connecting the fractionating columns with a condenser unit; 4) a condenser unit for condensing evaporated fractions; 5) a diverter valve for diverting flow of the condensed fractions into their respective receiving flasks; and 6) a plurality of receiving flasks for receiving the separated constituents.

In a fractional distillation of cannabinoids from a cannabis plant extract, three fractions are commonly distilled: 1) an organic solvent fraction; 2) a terpene fraction; and 3) cannabinoids containing fraction. As known, cannabinoids have high boiling points, which means that cannabinoid containing fractions are also very viscous. Applicant notes that CBD distillates often crystallize at room temperature, and THC distillates are often very viscous, having a consistency similar to that of heavy oil, honey or tar.

Accordingly, there is a need for fractional distillation system for the isolation and distillation of cannabinoids from a cannabis plant extract that can overcome the challenges presented by a highly viscous THC distillate or crystallization of CBD distillates.

### SUMMARY

A fractional distillation system can be used to isolate and collect cannabinoids from plant extracts, such as crude oil from plants. Fractional distillation systems comprises an external heat source for providing heat to vaporize the extract in a reaction vessel. The vapors travel through a fractionating column to separate the various constituents therein into their individual fractions. Each vaporized fraction is then condensed into a liquid distillate in a condensing head and is directed to be collected in appropriate receiving vessels. In order to maintain a fluidity of the liquid distillates, the liquid distillates are directed to flow through a thermal jacket. A diverter vale can be actuated by a processor to direct the liquid distillate to a particular receiving vessel.

In a broad aspect of the invention, a cannabis distillation apparatus can comprise a vertical fractionating column, a condensing head, and a thermal jacket whereby the condensing head is disposed on top of the fractionating column and the thermal jacket is spaced radially away from the condensing head.

In another broad aspect of the invention, a thermal jacket for a distillation apparatus can comprise an outer wall and an inner wall defining a jacket portion therebetween, an inlet, and an outlet providing access to the jacket portion adapted to receive a warm fluid.

In another broad aspect of the invention, a method for distilling cannabinoids can comprise the steps of vaporizing an extract containing the cannabinoids into vapors in a reaction vessel, fractionally distilling the vapors through a fractional distillation column, condensing the vapors into a plurality of liquid distillates, heating the plurality of liquid distillates to maintain fluidity thereof, monitoring the collection of the plurality of liquid distillates, directing each of the plurality of distillates to a plurality of receiving vessels, and collecting the plurality of liquid distillates.

Yet still, in another broad aspect of the invention, a control system for controlling fractional distillation of a plant extract containing cannabinoids, the control system can comprise a processor, at least one application programming interface, sensors, pumps, and at least one programmable logic controller.

### BRIEF DESCRIPTION OF THE DRAWINGS

FIG. 1 is a schematic representation of an embodiment of the present invention, illustrating a heater, a reaction vessel, fractionating column, a vertical condenser head, a thermal jacket, a diverter valve, receiving vessels, sensors for monitoring pressure, and a processor.

FIG. 2 is a side view of an embodiment of the present invention illustrating a fractionating column, a distillation head, a vertical condensing unit, a thermal jacket, a diverter valve and a plurality of receiving vessels; and

FIG. 3 is a side view of an embodiment of the present invention illustrating a vertical fractionating column, and a vertical condensing head.

### DETAILED DESCRIPTION

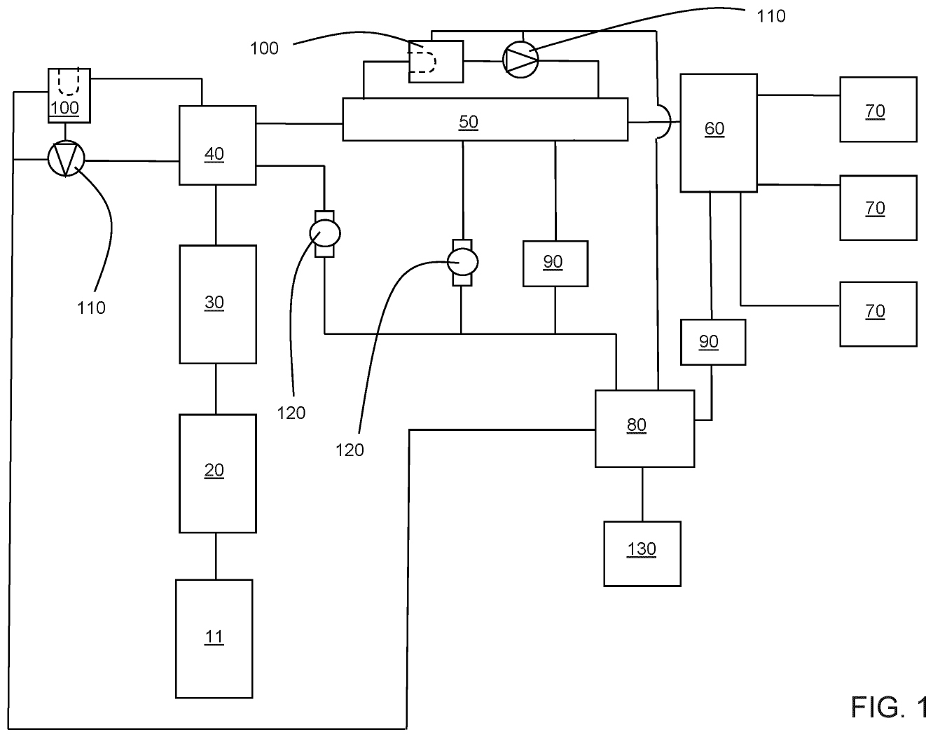

With reference to FIG. 1, an embodiment of the fractional distillation system 10 for distilling and isolating cannabinoids from plant extracts comprises a reaction vessel 20 or boiling flask in fluid communication with at least one vertical fractionating column 30. As known in the industry, the reaction vessel 20 can be heated by an external heat source 11 or can be a heated reaction vessel having heating elements incorporated therein. The reaction vessel 20 can comprise an open top portion which can be fluidly connected to an opened bottom end 31 of the vertical fractionating column 30.

In embodiments, the at least one vertical fractionating column 30 can be any kind of vertical fractionating column known in the art, such as an Oldershaw column, a straight column, or a Vigreux column. In embodiments, the vertical fractionating column 30 can be vacuum jacketed and/or silver lined for thermal insulation for maintaining consistent temperatures and thermal equilibrium within the fractionating column 30.

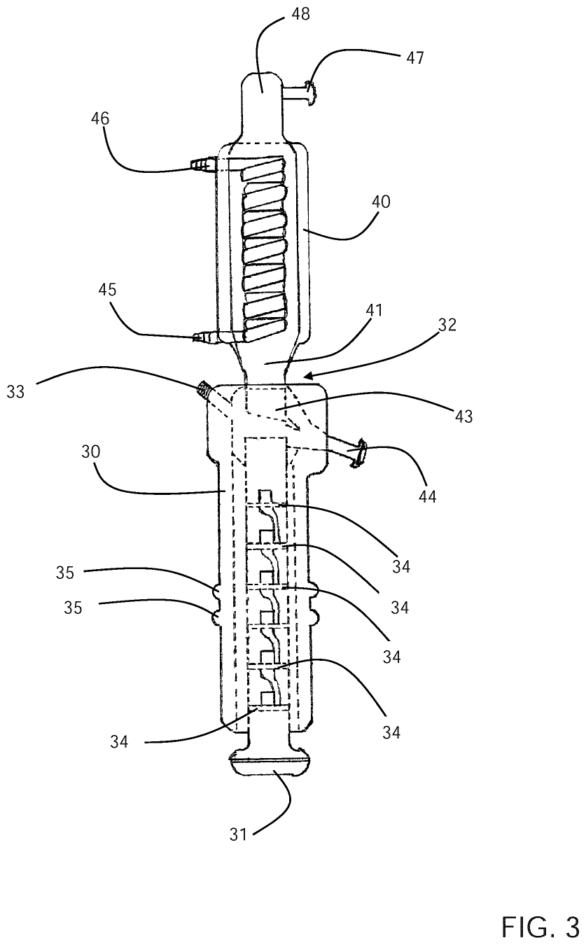

As shown in FIG. 1, the heat source 11 provides sufficient amounts of heat to vaporize any plant extracts and other additives such as solvents and other volatiles present in the reaction vessel 20. The vapors rising from the reaction vessel 20, enter into the fractionating column 30. As is well known in the art, the fractionating column separates the mixture of vapors coming off the reaction vessel 20 into individual and separate fractions, depending on the vaporization point of each component present. For example, vapor comprising a constituent that has a low vaporization point will be the first fraction to be separated from the plant extract and to reach the vertical condensing head 40, and to condense on a plate 34 (see FIG. 3). A subsequent fraction, having a vaporization point higher than the first fraction, will be the second fraction to be condensed by the condensing head 40, on a subsequent plate 34. A third fraction, having a vaporization point higher than the second fraction will be the third fraction separated and condensed, etc.

As shown, in an embodiment, the condensing head 40 is disposed on top of the fractionating column 30, and as the vapor rises through the fractionating column 20, the purified vapor (with several fractions removed as such as solvents, terpenes, etc) enters into the condensing head 40 where it is condensed and directed to a thermal jacket 50 that is disposed radially away from the condensing head 40. In an embodiment, the thermal jacket 50 is angled downwardly from the horizontal, optimally at an angle of about 68°, but optimally, from about 10° to 80°, angles wat which experimentation has determined that flow is optimal for a range of viscosities and jacket fluid temperatures. The condensed vapors or liquid distillates flow through the thermal jacket 50, through the diverter valve 60 and be directed to at least one receiving vessel 70 for collection and storage.

In embodiments, a processor 80 in conjunction with at least one programmable logic controller can automate the operation of the system 10. Sensors, such as pressure sensors 90 can monitor the overall pressure of the system 10 and detect changes in the operating pressure of the system during the collection of a particular fraction. The change in operating pressure can be an indicator of when a particular fraction has been distilled and collected. Upon detection of a change in the operating pressure, the processor 80 can then cause the heater 11 to increase the amount of heat provided to the reaction vessel 20 to cause a subsequent fraction to be vaporized in the fractionating column 30, condensed in the condensing head 40, and collected in a receiving vessel 70.

As shown, an operator can manipulate the conditions of the system 10 and re-program the processor 80 and at least one programmable logic controller using an application programming interface (API) 130. The API and the various sensors measures mantle temperature, vapor temperature, vacuum pressure, chiller temperature, valve positions and identifies which receiving vessel is collecting which fraction.

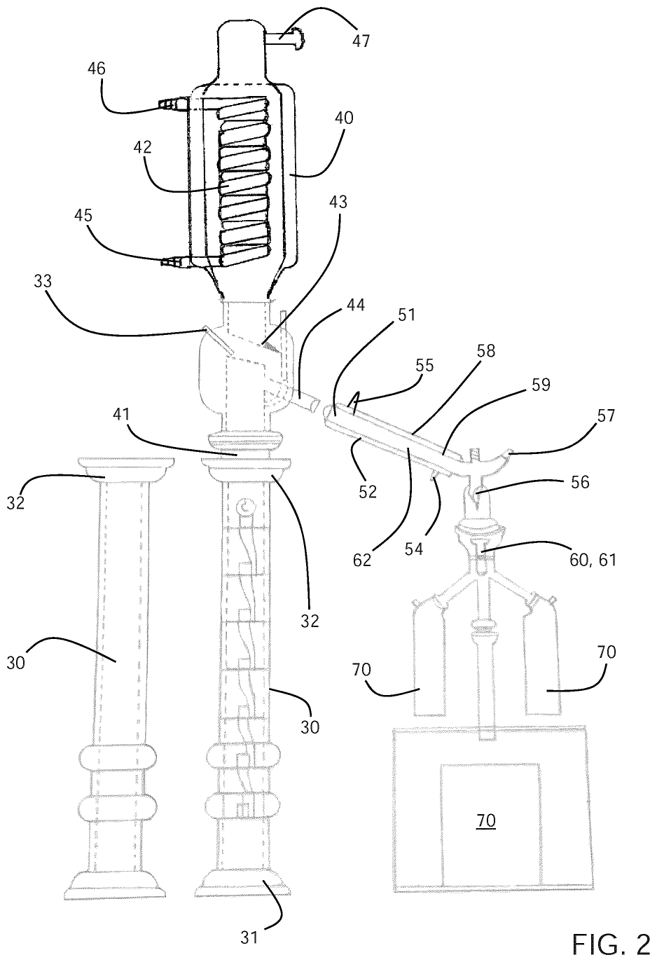

With reference to FIG. 2, an open bottom end 31 of the fractionating column 30 is operatively and fluidly connected to the open top end of the reaction vessel (not shown). An open top end 32 of the fractionating column 30 is operatively and fluidly connected to a bottom portion 41 of the condensing head 40. As shown, the condensing head 40 is disposed substantially on top of the fractionating column 30 in a vertical orientation, and further comprises a tubular helical coil 42 adapted to have a condensing fluid, such as water, flow therethrough. The flow of the condensing fluid through the tubular helical coil 42 functions as a heat exchange for the vapors in the vertical condensing head 40, decreasing a temperature of vapors within the vertical condensing head 40 to a threshold temperature below its vaporization point, and causing the vapors to condense into a liquid. The tubular helical coil 42 functions as a heat sink, and absorbs heat from the vapors, causing a temperature of the vapors to drop below the threshold temperature and to condense onto the tubular helical coil 42. The tubular helical coil 42 further provides an increased surface area for the heat exchange and an increased surface area for condensation to occur.

As shown, the tubular helical coil 42 has an inlet 45 and an outlet 46 that fluidly connects the helical coil 42 to a chiller 100, or any other known heat exchange device. The chiller 100 functions to provide a cool condensing fluid for use in the condensing head 40. Accordingly, a pump 110 can be incorporated into the system 100, if the chiller 100 does not already have a pump therein.

As shown, as well known in the art, the inlet 45 for the tubular helical coil 42 is positioned proximate to the bottom portion 41 of the condensing head 40, while the outlet 46 for the tubular helical coil 42 is positioned distal to the bottom portion 41 of the condensing head 40. This ensures that the tubular helical coil 42 is fully filled with the condensing fluid and that there are no dead spaces (spaces or portions of the tubular helical coil where there is no condensing fluid or air pockets inhibiting flow) within the tubular helical coil 42 where heat exchange may be negatively impacted.

In embodiments, the condensing head 40 can be vacuum jacketed and/or silver lined to assist in maintaining thermal insulation.

In further embodiments, and as discussed above, the fractionating column 30 and the condensing head 40 can be vacuum jacketed, or be under a vacuum pressure. As shown in FIG. 2, the condensing head 40 can be under vacuum pressure and accordingly has a vacuum port 47. Referring again to FIG. 1, the condensing head 40 can be operatively connected to a vacuum pump 120 through the vacuum port 47 (See FIG. 2).

Further still, in embodiments, and depending on the type of vertical fractionating column employed, such as a straight column, the vertical fractionating column 30 and the condensing head 40 can further be adapted to accept a helical band therethrough.

In typical fractional distillation systems known in the art, the vapors of each fraction are condensed in a condenser that is offset or positioned at an angle relative to the vertical fractionating column. The vapors coming off or rising from the fractionating column 30 travel to the top of the fractionating column 30 and then enter into the condenser that is positioned adjacent to or otherwise offset from the fractionating column. The design of the present invention avoids condensed liquids flowing back or re-entering the fractionating column from the condenser, as the condensed vapors travel away from the fractionating column. However, and with reference to FIG. 2, in an embodiment where the condenser is a vertical condensing head 40 and is positioned vertically above and disposed on top of the fractionating column 30, any condensed vapors in the vertical condensing head 40 can drip downwardly, and fall back into or re-enter the vertical fractionating column 30 thereunder, creating inefficiencies in processing. Accordingly, the present system redirects condensed vapors or liquid distillate away from the vertical fractionating column 30.

As shown, and in embodiments, to prevent condensed vapors or liquid distillate from falling through the vertical condensing head 40 and re-entering the fractionating column 30, the vertical condensing head 40 can comprise a drip tip 43, for redirecting condensed vapors towards an outlet 44 positioned adjacent the bottom portion 41 of the condensing head 40. The condensed vapors or liquid distillate fall or drip from the tubular helical coil 42 and are re-directed towards the outlet 44 by the drip tip 43. The drip tip 43 shape is key, in that it has a spatulate rear portion and a dorsal groove which capture liquid distillate and funnels the flow to the thermal jacket 50 in a flow efficient manner.

In an embodiment, and as shown, the outlet 44 is operatively and fluidly connected to a thermal jacket 50, such as a side-arm condensing unit, that is spaced radially away from the condensing head 40 and positioned at a downward angle from the horizontal. As shown, the thermal jacket 50 is offset the vertical condensing head 40 such that the thermal jacket 50 is fluidly connected to the vertical condensing head 40 at a downward angle from the horizontal, and having a downward slope to encourage the flow of the condensed vapors away from the condensing head 40. In embodiments, the downward angle of the thermal jacket 50 can be in the range of 10 degrees to 80 degrees. The liquid distillate falls from the drip tip 43 into the outlet 44, and is directed into a flow conduit 62 defined by an inner wall 59 of the thermal jacket 50, due to fluid pressure and gravity. The viscous nature of the liquid distillate is overcome, and the viscosity is lowered by the relatively high temperature of a heated fluid in the thermal jacket effecting the liquid distillate by thermal transfer (detailed below).

As shown, the outlet 44, adjacent the open bottom portion 41 of the condensing head 40, is adapted to be fluidly connected the flow conduit 62 at a proximal end 51 of the thermal jacket 50. Further, unlike the vertical condensing head 40, the thermal jacket 50 can be a double walled vessel and does not have a tubular helical coil disposed therein, but rather, the thermal jacket 50 can be a simple jacketed type condensing unit. The thermal jacket 50 has an outer wall 58 and an inner wall 59, defining a jacket portion 52 therebetween. As shown, an interior of the inner wall 59 further defines the flow conduit 62. The jacket portion 52 adapted to receive a warm or heated fluid flowing therethrough. The heated fluid provides heat to the liquid distillate travelling through the flow conduit 62, maintaining a temperature of the liquid distillate sufficiently high enough to lower a viscosity of the liquid distillate and keeping the liquid distillate fluid. As discussed above, any liquid distillate having a high viscosity can thicken and create obstacles that inhibit flow of the liquid distillate through the system, if not completely clog or block the system. Heating the liquid distillate travelling through the system is advantageous in that any liquid distillate coming off the condensing head 40 will remain fluid, preventing any blockages.

In an embodiment, the jacket portion 52 of the thermal jacket 50 accepts a heated fluid flow therethrough, instead of having the typical cooler condensing fluid flowing therethrough. The flow of a heated fluid through the jacket portion 52 permits an operator to use the thermal jacket 50 as a heat exchanger to keep the liquid distillate flowing through the thermal jacket 50 at a warm or heated temperature sufficiently high enough to keep the liquid distillate at a viscosity sufficiently low enough to enable the condensed vapors to flow relatively with ease. This is advantageous in circumstances where the condensed vapors inherently have a high viscosity, which may result in clogging of the distillation system, such as the case of cannabinoids. The use of the thermal jacket 50 allows an operator to finely control the operating temperature of the distillation system 10, and more specifically, the operating temperature of the liquid distillate of each of the plurality of fractions, thereby controlling the fluidity and flow rate of each of the liquid distillate fractions coming off the condensing head. Regardless of the fraction flowing through, the fluidity and the flow rate of the liquid distillate fraction or separated constituent can be controlled and regulated by controlling the temperature of the heated fluid flowing through the thermal jacket 50.

As shown, the thermal jacket 50 has an inlet 54 adjacent a distal end 53 of the thermal jacket 50, and has an outlet 55 adjacent the proximal end 51 of the thermal jacket 50. Skilled persons in the art would understand that such a layout of the inlet 54 and the outlet 55 ensures that the thermal jacket portion 52 is completely filled with the heated fluid and that there are no empty space that is not in contact with the heated fluid.

In embodiments, a drip tip 56 is positioned adjacent the distal end 53 of the thermal jacket 50 for directing all liquid distillates to a diverter valve 60. As shown, the flow conduit 62 of the thermal jacket is in fluid communication with the drip tip 56. Accordingly, liquid distillate enters and flows through the flow conduit 62 to exit the thermal jacket at a distal end thereof. The drip tip 56 redirects the liquid distillate flowing through the flow conduit 62 of the thermal jacket 50 towards at least one of a plurality of receiving flasks. As shown, the drip tip 56 further comprises an opening or vacuum port 57 adapted to fluidly connect to a vacuum suction source which can be provided by a vacuum pump 120. In embodiments, the drip tip 56 can be jacketed for insulation purposes.

In embodiments, the diverter valve 60 can be a jacketed diverter valve and can comprise an electromagnetic diverter valve 61. In operation, the electromagnetic diverter valve 61 can be controlled by a processor 80 and at least one programmable logic controller to automatically divert fractions collected such that each individual fraction can be diverted and collected in its respective receiving vessel 70. In embodiments, an operator can use an application programming interface (API) 130 to manipulate operating conditions and thresholds of the overall distillation process.

As shown, the diverter valve 60 redirects the liquid distillate flowing from the flow conduit 62 into a collection or receiving vessel 70. In embodiments, a plurality of receiving vessels 70 can be fluidly connected to the system 10, and as the processor determines a particular fraction that is coming off the thermal jacket 50, the processor 80 can actuate the diverter valve 60 to redirect the liquid distillate to be collected in a particular receiving vessel 70. In an embodiment, instead of having a movable diverter valve, the system 10 optionally comprises a carousel having a plurality of receiving vessels thereon. Accordingly, instead of actuating the diverter valve 60 for redirecting liquid distillate to its appropriate receiving vessel 70, the carousel can optionally be rotated by an operator or the control system so that the appropriate receiving vessel is aligned for receiving a particular liquid distillate. Both embodiments allow the system to be operated continuously with limited manual input by an operator, thereby reducing costs and increasing efficiency.

In a preferred embodiment, and with reference to FIG. 3, and in an embodiment, the system 10 is shown to have an Oldershaw vertical fractionating column 30 adapted to be operatively and fluidly connected to a reaction vessel (not shown). As shown, in an embodiment, the fractionating column 30 is disposed on top of the reaction vessel, and can have an open bottom end 31 for fluidly connecting to the reaction vessel. In preferred embodiments, the open bottom end 31 is about 51 mm in diameter and has an overall length of about 500 mm. Opposite the open bottom end 31 is an open top end 32 for fluidly connecting to a condensing head 40. Adjacent the top open end 32, the fractionating column 30 can comprise a vapor thermometer connection 33 for accepting a thermometer therethrough for measuring a temperature of the vapors within the fractionating column 30. In a preferred embodiment, the vapor thermometer connection 33 can be a GL type fitting sized appropriately for receiving a thermometer (not shown).

The fractionating column 30 can be optionally vacuum jacketed and silver-lined for thermal insulation, and comprises a plurality of plates 34. For cannabinoid resin mixed with solvents, the optimal number is a total of six (6) plates 34 spaced approximately 50 mm apart in height, with a dimpled collection of trays or a diameter of least 50 mm to enable sufficient vapor flow. In a preferred embodiment, each of the six (6) plates 34 are about 51 mm in diameter, can be spaced away from one another by a distance of about 51 mm. To further increase the number of theoretical plates/surface area of each plate, each plate has optimally 125 0.75 mm holes cut therethrough and incorporates a reflux channel for increasing efficiency of fractional distillation. This provides a column with more plates and surface area to separate compound that may be stuck together before they reach the head where they are condensed. Through trial and error, the Applicant has found that this particular sizing provides optimal thermal equilibrium and optimal overall performance of the system.

In embodiments, the fractionating column 30, while vacuum jacketed, can also incorporate expansion bellows 35 to provide additional room for expansion of air during the distillation process.

As shown, the open top end 32 of the fractionating column 30 is adapted to accept a bottom portion 41 of the vertical condensing head 40 so that the condensing head 40 is disposed on top of the fractionating column 30. A tubular helical coil 42 is positioned or disposed within the condensing head 40 to permit a condensing fluid, for instance, water, to flow therethrough from a heat exchanger, such as a chiller (see FIG. 1), for causing condensation of any vapors within the condensing head 40 onto a surface thereof. The condensing fluid can be pumped from the chiller (see FIG. 1) by a pump (see FIG. 1) through an inlet 45, through the tubular coil 42, and out through an outlet 46, back to the chiller and thus creating a circular flow path. The flow rate of the condensing fluid through the tubular coil 42 can be monitored by a processor (see FIG. 1) through the use of sensors (see FIG. 1), and controlled by the pump (see FIG. 1). In preferred embodiments, the condensing head 40 can have a total length of about 420 mm, wherein the bottom portion 41 thereof is about 150 mm in length, and the top portion 48 thereof is about 70 mm. Further still, in preferred embodiments, the inlet 45 and outlet 46 from the tubular coil can be ⅜ inches in diameter.

As previously discussed above, the system 10 can be operated under vacuum pressure in order to reduce the vaporization point of the desired constituents. Accordingly, the condensing head 40 can comprise a vacuum port 47 near a top portion 48 of the condensing head 40.

The vapors coming into contact with the surface of the tubular coil 42 will condense and form a liquid distillate. The liquid distillate will drop and fall through the condensing head 40 towards the fractionating column 30 thereunder. The condensing head 40 comprises a drip tip 43 positioned adjacent or near the bottom portion 41 of the condensing head 40 and redirects the liquid distillate towards an outlet 44 which is operatively and fluidly connected to a flow conduit 62 through a thermal jacket 50 (see FIG. 2). As shown, the thermal jacket 50 can be radially spaced away from the condensing head 40, and positioned at a downward angle of about 68° from the horizontal. This downward angle provides a sufficient slope to encourage flow of the liquid distillate from the condensing head 40, through thermal jacket 50 and towards the diverter valve 60. This sidearm must have a condenser on it in order to change temperature and avoid clogging of the distillate, especially while running remotely.

In an embodiment, a “winterized crude oil” of cannabis plant extract can be fractionally distilled to isolate cannabinoids, such as Δ-9-THC, THCV, Δ-8-THC, Δ-10-THC, CBD, CBDV, CBC, CBN.

Winterized crude oil is a full spectrum extract from cannabis or hemp biomass made with ethanol, hydrocarbon, carbon dioxide, water or mechanical extraction. The winterization process removes plant fats, waxes and lipids by freezing the solvent and cannabis solution over a period of time and then vacuum filtering, followed by separating the solvent. This would be sufficient to cause any solvents and volatiles present in the crude oil to be fractionally vaporized and enter the condensing head. Once the “crude extract” has been winterized it is loaded into the boiling flask (mantle flask or reaction vessel 20). The system is closed and the vacuum pump 120 is started. Once the vacuum pump 120 has been started, an initial pressure of 95 mmHg-115 mmHg is achieved and the heating mantle begins its heating cycle based off of a 10 second window “heat rate %” (50% =5 seconds on, 5 seconds off, 70% =7 seconds on, 3 seconds off, etc) and the stir bar is set, optimally, to 350 rpm and the head condenser and sidearm condenser are set to −20° C. to −10° C., the vacuum pressure is also lowered to 0.001 mmHg at this point. Once the solution begins to heat up, the residual solvent that may be left over in the crude begins to distill up the column and condense at the head, where it then falls off the head drip tip, out through the sidearm, where it drips off the drip tip into a designated receiving flask, through diverter valves or not. As the temperature rises, the solvent fraction will finish, the condensers are now adjusted to 20° to 25° C. and the terpenes and other volatile compounds will start to distill up through the column and condense in the condenser, follow the same route off the drip tip and through the sidearm through the diverter valves/or not, and drip down into a designated receiving flask. Once this fraction has finished, the heat will continue to rise until the cannabinoid fraction begins to start (D-9THC, THCV, D8THC, D10THC, CBD, CBDV, CBC, CBN), as the heating mantle rises, the cannabinoids will distill up the column, condense in the head condenser and follow the same route off the drip tip, through the sidearm, off the secondary drip tip and into a designated receiving flask. Once the distillation of the “crude” has been completed, the system backfill valve is opened and the machine reaches atmospheric equilibrium and is able to be opened where the different compounds are able to be collected. The residual solvent is a low viscosity like ethanol, the terpenes are liquid viscosity similar to water and a slight bit more viscous, while the cannabinoids are very viscous like honey or molasses.

Initially, the winterized crude oil is placed into the reaction vessel and the entire system is closed. The vacuum suction can be commenced at a vacuum pressure of between 95 mmHg to 115 mmHg, and heat can be applied to the reaction vessel containing the winterized crude oil. The heater can be set to a heating cycle based off a 10 second window providing heat at a 50% heat rate or a 70% heat rate. A 50% heat rate is defined as providing heat for 5 seconds, followed by 5 seconds of no heat. A 70% heat rate is defined as providing heat for 7 second followed by 3 seconds of no heat. A stir bar rotating at about 350 rpm is used to provide constant agitation of the winterized crude oil during the distillation process.

Once the heater is ready, the vacuum pressure of the system can be set to about 0.001 mmHg and the condensing fluid is at a temperature of about −20° C. to −10° C. As the heater constantly provides heat to the winterized crude oil, the solvents vaporize and travel through the fractionating column and come into contact with the surface of the tubular coil and condense thereon. The initial liquid distillate containing solvents drips downwardly towards the drip tip and is directed to the flow conduit of the thermal jacket. In embodiments, solvents and other volatiles often have low vaporization points and are inherently of low viscosity. Accordingly, a heated fluid need not be passed through the thermal jacket to maintain fluidity of the liquid distillate of the first fraction containing solvents.

The first fraction containing liquid distillates of the solvents are condensed and recovered in a first receiving vessel. Once the solvents are distilled and collected, the processor and at least one programmable logic controller will cause the heater (see FIG. 1) to increase the heat to the reaction vessel. The processor and at least one programmable logic controller also actuates the diverter valve (see FIG. 1) to direct the subsequent liquid distillate fraction (containing terpenes and other volatiles) to be collected in a second receiving vessel.

The increase in temperature to the reaction vessel and the fractionating column will cause a subsequent fraction containing terpenes to be fractionally distilled and collected. In preferred embodiments, the temperature of the condensing fluid during the distillation of the terpene fraction is between 20° C. to 25° C. As the liquid distillate containing terpenes inherently has low viscosity, a heated fluid need not be flowed through the thermal jacket.

In embodiments, once the terpene fraction is completely distilled and recovered, the processor detects any change in the pressure and accordingly adjusts the temperature of the heater in order to provide additional heat to the remaining crude oil for fractionally distilling a third fraction containing the desired cannabinoids. Once again, the processor will also actuate the diverter valve to redirect all upcoming liquid distillates to flow into and be collected in a third receiving vessel.

In embodiments, the system is further heated to a temperature sufficient to vaporize the desired cannabinoids, such as Δ-9-THC, THCV, Δ-8-THC, Δ-10-THC, CBD, CBDV, CBC, CBN) in the reaction vessel. The liquid distillate containing the desired cannabinoids inherently has a high viscosity and according, it is advantageous to flow a heated fluid through the thermal jacket when distilling the third fraction containing cannabinoids.

In embodiments, the system incorporates a variety of sensors in order to monitor vapor temperatures, vacuum pressures, mantle temperatures and other environmental factors to determine progress of the distillation, determine what fractions are coming off the system and a processor can be employed to control the overall progress of the distillation process. The various sensors would work in conjunction with processor control the overall operation of the system 10 and the distillation progress. In embodiments, an operator can change the parameters or operating conditions through an application programming interface (API). Accordingly, embodiments of the system can be operated remotely and autonomously. The automation of the system is written using the API, for example, accessed via a touchscreen controller, and uses inputs including mantle temperature, vapor temperature, and vacuum temperature. The processor is programmed to identify compounds based on the compound's vapor temperature at a pre-set vacuum pressure. Once a particular compound is identified, it is directed to a designated receiving vessel for collection and storage.

The touchscreen controller can also be used to operate the system manually.

In embodiments, the processor can record various data points, such as mantle temperature, vapor temperature, vacuum pressure, chiller temperature, valve positions, receiving vessels, and also record the distillation process visually through cameras. This permits an operator to very accurately remotely troubleshoot the system if necessary. The system can be accessed remotely for diagnostics and operation.

In embodiments, the API has a visual layout of the systems process control displaying various operational parameters, such as, for instance, mantle temperature, stir bar speed, thermal jacket chiller, flow conduit chiller, condenser head chiller, vapor temperature, diverter valve position, collection carousel vacuum pump on/off, vacuum pressure presets, led lights on/off, head to cold trap valve position/adjustment, thermal jacket to cold trap position/adjustment, collection carousel to cold trap position/adjustment, carousel backfill valve position/adjustment as well as system backfill/adjustment on a primary page.

On a secondary page of the touchscreen, the control touchscreen has presets for running automatically including all stated above variables in a spreadsheet layout. There is a cascade mode in an embodiment whereby the presets are followed and the system runs continuously and automatically. Cameras have been mounted on the unit for remote viewing of the mantle/reaction vessel, condensing head, receiving carousel, vacuum valve position, receiving carousel and drip tip of where the distillate comes through the flow conduit in the thermal jacket. In embodiments, the API also displays a real time data graph of all components listed above. To operate remotely, an operator can download a mobile application software to exert full control with a display on the device exactly like the one on the unit.

For example, in embodiments, the system 10 can be operated automatically using a processor and suitable software programs for controlling the heat applied to the reaction vessel, the flow rate and temperature of the condensing fluid flowing through the vertical condenser unit, the flow rate and temperature of the fluid flowing through the thermal jacket, the vacuum pressure applied to the entire system, and controlling the electromagnetic diverter valve. In an embodiment, the system an include pressure sensors to determine the vacuum pressure applied to the overall system and changes to the vacuum pressure applied to the system. The processor can then rely on the change in vacuum pressure to actuate the electromagnetic diverter valve for directing a distilled fraction to its appropriate receiving vessel.

Embodiments of the present invention are advantages because it is the first remote control, remote manageable distillation system in the cannabis industry. The combination of an Oldershaw column combined with a vertical condensing head creates more theoretical plates for use during fractional distillation, thus providing greater separation capability than what is currently available on the market, resulting in a much higher purity distillate in fewer passes. No other system on the market can currently be audited by regulatory bodies. No other distillation system has machine learning capabilities currently on the market as well, example, if there are 40 units in the field in operation, all 40 of them will feed data back to one central program that will learn and optimize distillation procedures.

In operation, embodiments of the present invention can be adapted to be operated under a vacuum pressure to decrease the boiling points of desired constituents, namely the organic solvents, terpenes and the cannabinoids.

1. A cannabis distillation apparatus having:
a vertical fractionating column;
a condensing head; and
a thermal jacket whereby the condensing head is disposed on top of the fractionating column and the thermal jacket is spaced radially away from the condensing head.

2. The apparatus of claim 1. wherein the thermal jacket is disposed radially away from the condensing head at a downward angle from the horizontal.

3. The apparatus of claim 2 wherein the downward angle is in the range of 10° to 80°.

4. The apparatus of claim 1, wherein the thermal jacket is a double walled vessel.

5. The apparatus of claim 4, wherein the double wall vessel comprises outer wall and an inner wall defining a jacket portion therebetween, the jacket portion adapted to receive a heated fluid therein.

6. The apparatus of claim 1, wherein the fractionating column is vacuum jacketed.

7. The apparatus of claim 1, wherein the fractionating column is silver lined.

8. The apparatus of claim 1, wherein the condensing head is vacuum jacketed.

9. The apparatus of claim 1, wherein the condensing head is silver lined.

10. A thermal jacket for a distillation apparatus, the thermal jacket comprising:
an outer wall and an inner wall defining a jacket portion therebetween;
an inlet; and
an outlet providing access to the jacket portion adapted to receive a warm fluid.

11. The thermal jacket of claim 10, further comprising a drip tip.

12. The thermal jacket of claim 10, further comprising a vacuum port.

13. A method for distilling cannabinoids comprising the steps of:
vaporizing an extract containing the cannabinoids into vapors in a reaction vessel;
fractionally distilling the vapors through a fractional distillation column;
condensing the vapors into a plurality of liquid distillates;
controlling fluidity of each of the plurality of liquid distillates;
heating the plurality of liquid distillates to maintain fluidity thereof;
monitoring the collection of the plurality of liquid distillates; directing each of the plurality of distillates to a plurality of receiving vessels; and
collecting the plurality of liquid distillates.

14. The method of claim 13, wherein controlling the fluidity of the plurality of liquid distillates further comprises heating the plurality of liquid distillates.

15. The method of claim 14, wherein heating the plurality of liquid distillates further comprising passing the plurality of liquid distillates through a thermal jacket.

16. The method of claim 15, wherein passing the plurality of liquid distillates through a thermal jacket further comprises flowing a heated fluid through the thermal jacket.

17. The method of claim 13, wherein condensing the vapors further comprises passing a condensing fluid through a condensing head.

18. The method of claim 13, wherein directing each of the plurality of liquid distillates to a plurality of receiving vessels further comprises actuating a diverter valve.

19. The method of claim 13, wherein directing each of the plurality of liquid distillates to a plurality of receiving vessels further comprises rotating at least two of the plurality of receiving vessels.

20. (canceled)
